(19) United States
(12) Patent Application Publication
(10) Pub. No.: US 2026/0142010 A1
(43) Pub. Date: May 21, 2026

(54) LINEAR MODEL-BASED ADAPTIVE CLOSED-LOOP CONTROL METHOD

(71) Applicant: SHANDONG SITURING CO., LTD., Jinan (CN)

(72) Inventor: Can PENG, Jinan (CN)

(73) Assignee: SHANDONG SITURING CO., LTD., Jinan (CN)

(21) Appl. No.: 19/121,453

(22) PCT Filed: Jun. 7, 2023
(86) PCT No.: PCT/CN2023/099003

§ 371 Date: Apr. 16, 2025

(30) Foreign Application Priority Data
Oct. 19, 2022 (CN) 202211281716.4

(51) Int. Cl. G16H 20/17; A61M 5/172

(52) U.S. Cl. CPC G16H 20/17; A61M 5/1723; A61M 2230/201

(57) ABSTRACT

Some embodiments provide linear model-based adaptive closed-loop control methods. In some examples, the method includes: acquiring preset parameters, acquiring the target physiological parameter of the object and a residual activity parameter of the chemical substance, calculating a sensitivity coefficient of the chemical substance based on the target physiological parameter and the residual activity parameter, calculating an increase rate coefficient of the target physiological parameter based on the target physiological parameter and the residual activity parameter, the increase rate coefficient characterizing an influence level of factors other than the chemical substance on the target physiological parameter, calculating a safe dose and a basal dose of the chemical substance based on the preset parameters, the target physiological parameter, the residual activity parameter, the sensitivity coefficient and the increase rate coefficient, and calculating a target dose of the chemical substance based on the safe dose and the basal dose.

### CROSS REFERENCE TO RELATED APPLICATIONS

This application is the United State national stage entry under 37 U.S.C. 371 of PCT/CN2023/099003, filed on Jun. 7, 2023, which claims priority to Chinese application number 202211281716.4, filed on Oct. 19, 2022, the disclosure of which are incorporated by reference herein in their entireties.

### FIELD OF THE DISCLOSURE

The disclosure relates generally to medical devices. More specifically, the disclosure relates to linear model-based adaptive closed-loop control methods.

### BACKGROUND

Diabetes is a metabolic disease characterized by hyperglycemia, for diabetes patients, they have symptoms such as hyperglycemia, unstable blood glucose and dyspepsia due to an insulin secretion defect and/or an impaired biological function. In this case, chronic damage will be brought to various kinds of tissue (especially eyes, kidney, heart, blood vessels and nerves). In some cases, the diabetes patients will be treated by taking medicines to relieve symptoms. For example, type 1 diabetes patients will be injected with insulin to avoid the hyperglycemia, and some type 1 diabetes patients will also use an insulin pump for adjuvant treatment, however, since an increase or decrease of blood glucose is related to various factors, it is difficult to accurately control a change of the blood glucose. For a treatment method of injecting the insulin, it is easy to cause continuous hyperglycemia due to insufficient injected volume or hypoglycemia due to excessive injected volume, thereby endangering the health of the diabetes patients. During medicine taking, in order to reduce untoward reactions caused by overdose or underdose of the medicine, the dose of the medicine will be regulated using a closed-loop algorithm.

At present, commonly used closed-loop algorithms include a conventional PID algorithm or an action model algorithm. It is necessary for the conventional PID algorithm to input multiple parameters with uncertain physiological significance, and therefore, a personalized adjustment is required, while it is necessary for the action model algorithm to input intaking carbohydrate that is difficult to accurately quantify. For example, a Chinese patent application with a publication number of CN113453619A discloses a safety tool to make decision support recommendations for a user of a continuous glucose monitoring system, which requires to receive multiple input data terms that affect a diabetes condition of a user of a continuous glucose monitor. The multiple input data terms include nutrition data of a food or drink (equivalent to the intaking carbohydrate). Therefore, the current closed-loop algorithm for regulating the dose of the medicine still has a relatively high threshold for use, and the input of intaking carbohydrate that is difficult to accurately quantify is not conducive to daily use of an ordinary user.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, the present disclosure proposes a linear model-based adaptive closed-loop control method, which is a control method to control a target physiological parameter of an object using a chemical substance, and includes: acquiring preset parameters, acquiring a target physiological parameter of the object and a residual activity parameter of the chemical substance, calculating a sensitivity coefficient of the chemical substance based on the target physiological parameter and the residual activity parameter, the sensitivity coefficient being used for characterizing an influence level of the chemical substance on the target physiological parameter, calculating an increase rate coefficient of the target physiological parameter based on the target physiological parameter and the residual activity parameter, the increase rate coefficient being used for characterizing an influence level of factors other than the chemical substance on the target physiological parameter, calculating a safe dose and basal dose of the chemical substance based on the preset parameters, the target physiological parameter, the residual activity parameter, the sensitivity coefficient and the increase rate coefficient, and calculating a target dose of the chemical substance based on the safe dose and the basal dose.

In this case, the sensitivity coefficient and the increase rate coefficient may be adaptively adjusted, which improves control accuracy. At the same time, since the increase rate coefficient may be used for characterizing the influence level of the factors other than the chemical substance on the target physiological parameter, and the increase rate coefficient is obtained by calculation of the target physiological parameter and the residual activity parameter, resulting in reducing input parameters (such as the carbohydrate intake of a subject), and then, lowering the threshold for use of the adaptive closed-loop control method.

In addition, in an adaptive closed-loop control method according to an embodiment of the disclosure, optionally, the preset parameters include a physiological information of the object, a type of the chemical substance, a control step size, an unit of the target physiological parameter, a safety threshold value of the target physiological parameter, and an infusion accuracy of the chemical substance. In this case, the parameters may be adaptively adjusted in advance according to the preset parameters, which improves the control accuracy.

In addition, in an adaptive closed-loop control method according to an embodiment of the disclosure, optionally, the preset parameters are obtained by data import or manual input. In this case, the preset parameters may be conveniently obtained by data import, which further lowers a threshold for use. The preset parameters may be conveniently modified by manual input.

In addition, in an adaptive closed-loop control method according to an embodiment of the disclosure, optionally, the chemical substance of the infusion dose is enabled to enter the object by infusion. The infusion dose is related to the target dose and the infusion accuracy. In this case, the calculated target dose may cooperate with actual infusion accuracy of an actual device to obtain the infusion dose that may be infused to the target within an accuracy range.

In addition, in an adaptive closed-loop control method according to an embodiment of the disclosure, optionally, the infusion dose is obtained by metering of an infusing device. In this case, due to uncertainty of actual infusion, the infusion dose is obtained using metering of the infusing device, so that the actual dose of the chemical substance infused into the object may be more accurately obtained, it is facilitated to improve the accuracy of subsequent regulation.

In addition, in an adaptive closed-loop control method according to an embodiment of the disclosure, optionally, the target physiological parameter is collected by a continuous blood glucose monitoring apparatus or a blood glucose meter. In this case, the target physiological parameter may be acquired in different ways.

In addition, in an adaptive closed-loop control method according to an embodiment of the disclosure, optionally, the target physiological parameters of the object at a plurality of time nodes and the residual activity parameter of the chemical substance are acquired, the plurality of time nodes include a target node and other nodes, and the residual activity parameter of the target node is obtained based on the residual activity parameters of the other nodes. In this case, parameters such as the residual activity parameter, the increase rate coefficient, or the sensitivity coefficient will not change too much in a short time. When parameters such as a residual activity parameter, an increase rate coefficient, or a sensitivity coefficient of one time node are calculated, the accuracy of calculation may be improved using parameters of a plurality of time nodes.

In addition, in an adaptive closed-loop control method according to an embodiment of the disclosure, optionally, the sensitivity coefficient located at the target node is obtained by minimizing a loss function. The loss function includes an error term and a regularization term. The error term is used for characterizing a difference between the target physiological parameter acquired by measurement and the target physiological parameter acquired by calculation. The regularization term is used for characterizing a difference degree of the sensitivity coefficients of adjacent time nodes. In this case, the accuracy of the sensitivity coefficient of each time node may be improved using an error term. At the same time, since the sensitivity coefficient will not change too much in a short time under a normal circumstance, the stability and accuracy of the sensitivity coefficient may also be improved using the regularization term.

In addition, in an adaptive closed-loop control method according to an embodiment of the disclosure, optionally, the sensitivity coefficient of the target node is acquired based on the sensitivity coefficients of the other nodes, the target physiological parameters of the other nodes, the residual activity parameters of the other nodes, and the target physiological parameter of the target node. In this case, the sensitivity coefficient of the target node may be calculated using some of parameters acquired in previous time nodes, thereby adaptively adjusting the sensitivity coefficient. At the same time, since the sensitivity coefficient will not change too much in the adjacent time nodes, the stability and accuracy of the sensitivity coefficient may be improved using the adjacent time nodes.

In addition, in an adaptive closed-loop control method according to an embodiment of the disclosure, optionally, the increase rate coefficient of the target node is obtained based on the difference between the target physiological parameter acquired by measurement and the target physiological parameter acquired by calculation located at the target node, and the increase rate coefficients of the other nodes. In this case, the increase rate coefficient of the target node may be calculated using some of parameters acquired in previous time nodes, thereby adaptively adjusting the increase rate coefficient, so that the accuracy of the sensitivity coefficient may be improved.

### BRIEF DESCRIPTION OF THE DRAWINGS

Illustrative embodiments of the disclosure are described in detail below with reference to the attached drawing figures.

FIG. 1 is a schematic diagram illustrating an application scenario of a linear model-based adaptive closed-loop control method according to an embodiment of the disclosure.

FIG. 2 is a schematic diagram illustrating a two compartment dynamics model according to an embodiment of the disclosure.

FIG. 3 is a flow chart illustrating a linear model-based adaptive closed-loop control method according to an embodiment of the disclosure.

FIG. 4 is a schematic diagram illustrating an application scenario of acquiring a target physiological parameter of an object according to an embodiment of the disclosure.

FIG. 5 is a curve diagram illustrating a target physiological parameter according to an embodiment of the disclosure.

FIG. 6 is a structural block diagram illustrating a linear model-based adaptive closed-loop control system according to an embodiment of the disclosure.

FIG. 7 is a flow chart illustrating a linear model-based adaptive closed-loop control system according to an embodiment of the disclosure.

FIG. 8a is a simulation schematic diagram illustrating that a plain PID method is applied to an adult according to an embodiment of the disclosure.

FIG. 8b is a simulation schematic diagram illustrating that a PIDIFB method is applied to an adult according to an embodiment of the disclosure.

FIG. 8c is a simulation schematic diagram illustrating that an adaptive closed-loop control method is applied to an adult according to an embodiment of the disclosure.

FIG. 9a is a simulation schematic diagram illustrating that a plain PID method is applied to an adolescent according to an embodiment of the disclosure.

FIG. 9b is a simulation schematic diagram illustrating that a PIDIFB method is applied to an adolescent according to an embodiment of the disclosure.

FIG. 9c is a simulation schematic diagram illustrating that an adaptive closed-loop control method is applied to an adolescent according to an embodiment of the disclosure.

FIG. 10a is a simulation schematic diagram illustrating that a plain PID method is applied to a child according to an embodiment of the disclosure.

FIG. 10b is a simulation schematic diagram illustrating that a PIDIFB method is applied to a child according to an embodiment of the disclosure.

FIG. 10c is a simulation schematic diagram illustrating that an adaptive closed-loop control method is applied to a child according to an embodiment of the disclosure.

FIG. 11 is a simulation schematic diagram illustrating that an optimized adaptive closed-loop control method is applied to a child according to an embodiment of the disclosure.

FIG. 12a is a simulation schematic diagram illustrating that an adaptive closed-loop control method with infinite infusion accuracy is applied to an adult according to an embodiment of the disclosure.

FIG. 12b is a simulation schematic diagram illustrating that an adaptive closed-loop control method with finite infusion accuracy is applied to an adult according to an embodiment of the disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

Hereinafter, embodiments of the present disclosure will be described in detail with reference to the accompanying drawings. In the following descriptions, the same reference numerals are given to the same parts, and repeated descriptions are omitted. In addition, the drawings are merely schematic, and the proportions of dimensions of the parts relative to one another or the shape of the parts etc. may differ from practical conditions.

It should be noted that the terms “include” and “have”, and any variations thereof in the present disclosure, such as a process, method, system, product, or device that includes or has a list of steps or elements are not necessarily limited to those explicitly recited steps or elements, but may include or have other steps or elements not explicitly recited or inherent to such process, method, product, or device.

It should be noted that the terms of relative positions and relative directions thereof in this article, such as “upside”, “toward upside”, “downside”, “toward downside”, “up and down direction”, “left side”, “toward left side”, “left”, “toward left”, “right side”, “toward right side”, “right”, “toward right”, “left and right direction”, “ahead”, “toward ahead”, “rear”, “toward rear” etc. are reference to the usually operational postures and should not be considered restrictive.

An embodiment of the present disclosure relates to a linear model-based adaptive closed-loop control method, which represents and characterizes an influence level of factors other than a chemical substance on a target physiological parameter using an increase rate coefficient, and meanwhile, adaptively adjusts a sensitivity coefficient and the increase rate coefficient. In this case, a physiological parameter related to the target physiological parameter may be adaptively adjusted, and input parameters are reduced.

An embodiment of the present disclosure relates to a linear model-based adaptive closed-loop control system, which control a target physiological parameter of an object using the adaptive closed-loop control method according to an embodiment of the disclosure. In this case, a physiological parameter related to the target physiological parameter may be adaptively adjusted, the stability of the target physiological parameter of the object is improved, and input parameters are reduced.

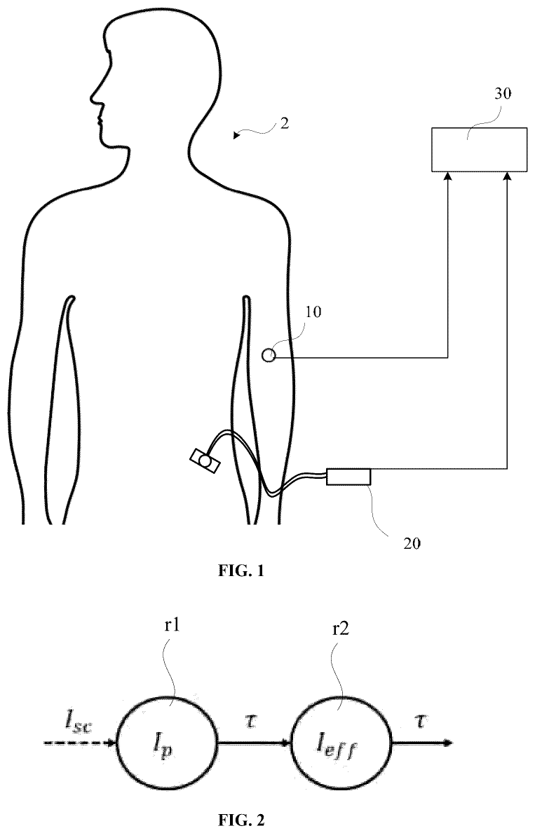

FIG. 1 is a schematic diagram illustrating an application scenario of a linear model-based adaptive closed-loop control method according to an embodiment of the disclosure.

In some examples, referring to FIG. 1, the linear model-based adaptive closed-loop control method may be a control method to control a target physiological parameter of an object 2. In some examples, the linear model-based adaptive closed-loop control method involved herein may be referred as an LMPID method, a control method, or an adaptive closed-loop control method.

In some examples, an infusion dose of the chemical substance may be obtained based on the adaptive closed-loop control method, and the chemical substance is infused to an object 2 by an infusing device 20. However, the present disclosure is not limited to this, the chemical substance may also enter the object 2 in multiple ways. For example, according to constituents of the chemical substance, the chemical substance may also enter the object 2 by oral administration, sublingual administration, rectal administration, mucocutaneous administration, inhalation administration, injection administration, etc. Optionally, the chemical substance may be infused to the object 2 by the infusing device 20. In this case, the chemical substance may be delivered to the object 2 rapidly, and the take-effect time of the chemical substance is accelerated.

In some examples, the infusing device 20 may be an infusing device 20 fixed to the object 2, and automatic infusion is performed based on the infusion dose of the chemical substance acquired by the adaptive closed-loop control method. In some examples, the infusing device 20 may also be a needle tubing. The chemical substance may be manually infused into the object 2 or other patients based on the infusion dose of the chemical substance acquired by the adaptive closed-loop control method. Optionally, the infusing device 20 may be an insulin pump (or an artificial pancreas). In this case, the chemical substance (e.g., insulin) may be conveniently delivered, and meanwhile enabling to be combined with a continuous glucose monitoring apparatus 10a (CGM) and a processing apparatus 30 to form a closed-loop insulin pump system.

In some examples, the object 2 may be an animal. For example, the object 2 may be a creature such as a human, an orangutan or a mouse. In some examples, the object 2 may be a patient with an endocrine disease. For example, the object 2 may be a patient with a defective endocrine gland (adrenal gland, thyroid gland, pancreas or pituitary gland). In this case, since the patient with the endocrine disease may have an abnormal secretory function and/or structure of the endocrine gland or endocrine tissue itself, an imbalance of a chemical substance used for regulation in the body occurs, thus leading to an imbalance of the target physiological parameter. The chemical substance may be controlled using the adaptive closed-loop control method according to an embodiment of the disclosure, and then, the target physiological parameter may be controlled within an appropriate range.

In some examples, the object 2 may be a patient with diabetes, hypopituitarism, a thyroid disease, or obesity. Optionally, the object 2 may be a patient with the diabetes, particularly type 1 diabetes (insulin dependent diabetes) patients. In this case, since the insulin is difficult to produce in the type 1 diabetes patient, hyperglycemia or large blood glucose fluctuation may be easily caused. The blood glucose may be effectively stabilized using the adaptive closed-loop control method according to an embodiment of the disclosure.

In some examples, the chemical substance may be a substance with a special effect on the body that is directly secreted into blood by human and animal endocrine organs or tissue. For example, the chemical substance may be a hormone (including: the chemical substance may be a pituitary hormone, insulin, glucagon, calcitonin, a parathyroid hormone, etc.), optionally, the chemical substance may be insulin.

In some examples, the chemical substance may be a synthetic hormone. In some examples, the chemical substance may be a substance that have a regulation effect on the blood glucose.

In some examples, the target physiological parameter may refer to a blood glucose concentration of the object 2. In some examples, the target physiological parameter may also refer to the substance in the blood that has the special effect on the body. For example, the target physiological parameters may also be some of hormones related to target physiological health.

Hereafter, in order to better describe the adaptive closed-loop control method, the adaptive closed-loop control method according to an embodiment of the disclosure is described by taking the object 2 as the diabetes patient, the chemical substance as insulin, and the target physiological parameter as the blood glucose concentration as an example. However, it should be noted that the adaptive closed-loop control system according to an embodiment of the disclosure is not limited to this. The adaptive closed-loop control system according to an embodiment of the disclosure is also applicable to other cases where the target physiological parameter of the object 2 are controlled using the chemical substance.

FIG. 2 is a schematic diagram illustrating a two compartment dynamics model according to an embodiment of the disclosure.

In some examples, the adaptive closed-loop control method may be based on a blood glucose dynamics model. In some examples, the blood glucose dynamics model may be expressed as:

$$Δ G ( k + 1 ) = G ( k + 1 ) - G ( k ) = φ {I}_{eff} + α$$

Wherein ΔG(k+1) represents a change of the target physiological parameter at a k+1^{th} time node (i.e., the change of the blood glucose concentration), G(k+1) represents the target physiological parameter at the k+1^{th} time node (i.e., the blood glucose concentration), φ represents the sensitivity coefficient (i.e., an insulin sensitivity coefficient) of the chemical substance, I_{eff} represents the residual activity parameter of the chemical substance (i.e., active insulin), and α represents the increase rate coefficient of the target physiological parameter.

In some examples, the blood glucose dynamics model is a linear model, so that the adaptive closed-loop control method may also be called the linear model-based adaptive closed-loop control method. In this case, since the linear model may conveniently reflect a rule of the blood glucose dynamics model at the adjacent time nodes (a short time interval), the calculation cost may be reduced, and the calculation speed is increased, and thus the timeliness of the adaptive closed-loop control method may be improved.

In some examples, a metabolic model of the insulin in the object 2 may be established using a two-compartment model. In some examples, referring to FIG. 2, the two-compartment model may include a central compartment r1 and an outer peripheral compartment r2. Wherein the insulin in the central compartment r1 may be insulin that is infused intravenously and enters the compartment. The insulin in the outer peripheral compartment r2 may be insulin that diffuses from the compartment into blood vessels, which may also be known as the active insulin.

In some examples, the two-compartment model may be expressed as:

$$\frac{d {I}_{p}}{d t} = {I}_{s c} - \frac{{I}_{p}}{τ} , \frac{d {I}_{eff}}{d t} = \frac{{I}_{p}}{τ} - \frac{{I}_{eff}}{τ} , h ( t ) = \frac{1}{{τ}^{2}} t {e}^{- \frac{t}{τ}} ,$$

Wherein t may represent time, Iₚ may represent the amount of the insulin in the central compartment r1, I_{sc} may represent the infusion dose of the insulin (i.e., the insulin infused to the object), τ may represent time that insulin reaches a peak, and h (t) may represent an impact response of the system.

In some examples, based on the two-compartment model, the metabolic model may be expressed as:

$${I}_{eff} ( k + 1 ) = {K}_{0} {I}_{s c} ( k ) + {K}_{1} {I}_{eff} ( k ) - {K}_{2} {I}_{eff} ( k - 1 ) , {K}_{1} = 2 {e}^{\frac{Δ t}{τ}} , {K}_{2} = {e}^{\frac{2 Δ t}{τ}} , {K}_{0} = 1 - {K}_{1} + {K}_{2} ,$$

Wherein k may represent a k^{th} time node, Δt may represent a time interval between two adjacent time nodes, Δt may also be called a control step size, and K₀, K₁ and K₂ may represent parameters related to diffusion rates of the insulin at different positions.

In some examples, K₀, K₁ and K₂ may be constants obtained on basis of the control step size and the time that the insulin reaches the peak, in other words, K₀, K₁ and K₂ can be obtained by the preset parameters.

In some examples, as described above, the adaptive closed-loop control method may be established based on the blood glucose dynamics model and the metabolic model.

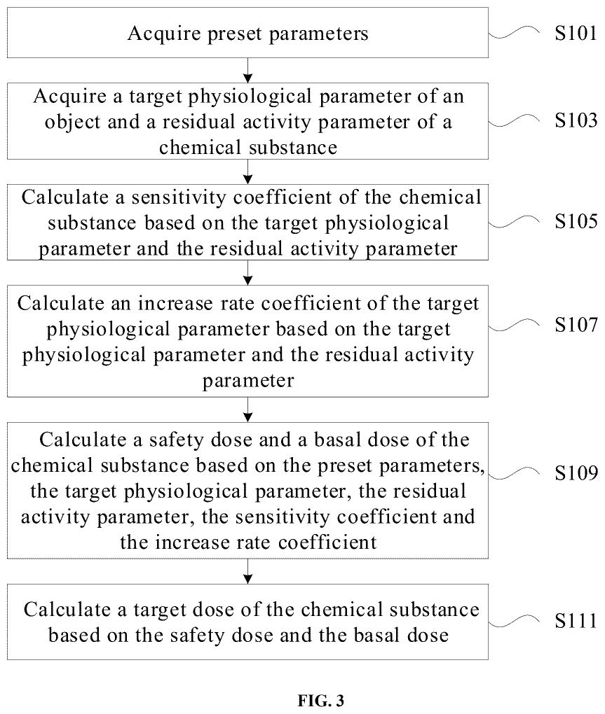

FIG. 3 is a flow chart illustrating a linear model-based adaptive closed-loop control method according to an embodiment of the disclosure.

In some examples, referring to FIG. 3, the adaptive closed-loop control method may include: acquiring the preset parameters (step S101), acquiring the target physiological parameter of the object 2 and residual activity parameter of the chemical substance (step S103), calculating a sensitivity coefficient of the chemical substance based on the target physiological parameter and the residual activity parameter (step S105), calculating an increase rate coefficient of the target physiological parameter based on the target physiological parameter and the residual activity parameter (step S107), calculating a safe dose and a basal dose of the chemical substance based on the preset parameters, the target physiological parameter, the residual activity parameter, the sensitivity coefficient and the increase rate coefficient (step S109), and calculating a target dose of the chemical substance based on the safe dose and the basal dose (step S111).

In this case, the sensitivity coefficient and the increase rate coefficient may be adaptively adjusted, which improves control accuracy. At the same time, since the increase rate coefficient may be used for characterizing the influence level of the factors other than the chemical substance on the target physiological parameter, and the increase rate coefficient is obtained by calculation of the target physiological parameter and the residual activity parameter, thereby reducing input parameters (such as the carbohydrate intake of the object 2), and then, the threshold for use of the adaptive closed-loop control method is lowered.

### (Step S101)

In some examples, as described above, in step S101, the preset parameters may be acquired.

In some examples, the preset parameters may include at least one of the physiological information of the object 2, the type of the chemical substance, the control step size, the unit of the target physiological parameter, the safety threshold value of the target physiological parameter, and the infusion accuracy of the chemical substance. In this case, the parameters may be adaptively adjusted in advance according to the preset parameters, which improves the control accuracy.

In some examples, the preset parameters may include the physiological information of the object 2. In some examples, the physiological information may include gender, age, weight, course of disease and other easy-to-acquire medical information. In some examples, the physiological information may include a family history and a medication history. In this case, since the sensitivity coefficient of the chemical substance in different objects 2 is highly correlated with the personal physiological information of the object 2, an initial value of the sensitivity coefficient may be estimated by acquiring the physiological information.

In some examples, the preset parameters may include the type of the chemical substance. For example, the preset parameters may include the type of the insulin. In this case, since different types of chemical substances have different time to reach the peak in the blood, it is able to set the time for the chemical substance to reach the peak in the blood based on the type of the chemical substance.

In some examples, the preset parameters may include the control step size, which may be an interval between two adjacent time nodes. In this case, the control step size is input, which may adjust the time node interval, so that different control step sizes may be selected for different objects 2. For example, if a fluctuation of the target physiological parameter of the object 2 is relatively stable, a larger control step size may be selected as the control step size. If a fluctuation of the target physiological parameter of the object 2 is relatively severe, a shorter control step size may be selected as the control step size. In some examples, an appropriate control step size may also be selected for other reasons.

In some examples, the preset parameters may include the unit of the target physiological parameter. For example, in a case where the target physiological parameter is the blood glucose concentration, the target physiological parameter may be millimole per liter (mmol/l) or milligram per deciliter (mg/dl). In this case, due to different usage habits, people in different regions may use different units. Therefore, the unit of the target physiological parameter is input, which may facilitate the usage habits of different people, and reduces the difficulty of conversion, so that the versatility of the adaptive closed-loop control method may be improved.

In some examples, the preset parameters may include the safety threshold value of the target physiological parameter. In this case, when the infusion dose of chemical substance is acquired subsequently, the appropriate infusion dose of the chemical substance may be obtained based on the safety threshold value, so that the target physiological parameter may be controlled within a safety range.

In some examples, the preset parameters may include the infusion accuracy of the chemical substance. In this case, since different infusing devices 20 may have different infusion accuracy, a same infusing device 20 may be also set to have different infusion accuracy, and the dose of the chemical substance actually infused into the object 2 is related to the infusion accuracy of the infusing device 20, which may not be exactly the same as the target dose calculated by the adaptive closed-loop control method. By acquiring the infusion accuracy of the chemical substance, the accuracy of the chemical substance actually infused into the object 2 may be determined, and then the dose of the chemical substance actually infused into the object 2 may be better determined, thus facilitating the calculation of the adaptive closed-loop control method.

In some examples, the preset parameters are obtained by data import or manual input. For example, a part of the preset parameters may be obtained by data import, and a part of the preset parameters may be obtained by manual input. In this case, the preset parameters may be conveniently obtained by data import, which further lowers a threshold for use. The preset parameters may be conveniently modified by manual input.

In some examples, some of the preset parameters may be input by medical personnel. In this case, the medical personnel may set the preset parameters based on professional knowledge and experience. In some examples, the preset parameters may also be input by the object 2. In this case, the object 2 may adaptively adjust the preset parameters based on an individual condition.

In some examples, some of the preset parameters may be obtained through internal parameters of the infusing device 20, for example, by calling the internal parameters of the infusing device 20, or through the technical specifications or an instruction for use of the device.

### (Step S103)

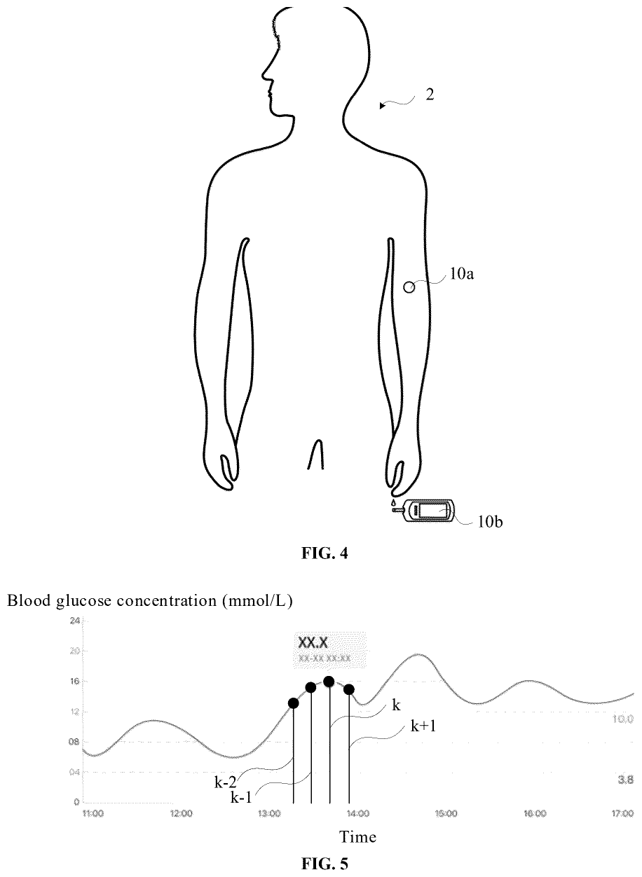

FIG. 4 is a schematic diagram illustrating an application scenario of acquiring a target physiological parameter of an object 2 according to an embodiment of the disclosure. FIG. 5 is a curve diagram illustrating a target physiological parameter according to an embodiment of the disclosure.

In some examples, step S103 may be acquiring the target physiological parameter of the object 2 and the residual activity parameter of the chemical substance. In this case, it is able to determine the residual activity parameter of the chemical substance. Since there may be the chemical substance still remaining active in the object 2, when the target dose is acquired using the adaptive closed-loop control method, the residual activity parameter of the chemical substance may be taken into consideration, which may reduce the situation that the target physiological parameter exceeds the safety range due to an excessive target dose (for example, the blood glucose concentration is lower than the safety threshold value).

In some examples, the residual activity parameter of the chemical substance may be understood in the following way: the residual activity parameter is the amount (or concentration) of the chemical substance remaining active in the object 2, or the amount (or concentration) of the chemical substance that may still take effect and adjust the target physiological parameter.

In some examples, referring to FIG. 4, the target physiological parameter may be obtained by the continuous glucose monitoring apparatus 10a (CGM) or a blood glucose meter 10b. In other words, an acquiring device 10 may be the continuous glucose monitoring device 10a or the blood glucose meter 10b. In this case, the target physiological parameter may be acquired in various ways.

In some examples, the target physiological parameter may be continuously acquired using the continuous blood glucose monitoring apparatus 10a. In this case, the continuous blood glucose monitoring apparatus 10a is used, which may improve the convenience of acquiring the target physiological parameter, and meanwhile it is facilitated for the processing apparatus 30 to process a mass of target physiological parameters. In some examples the continuous blood glucose monitoring apparatus 10a may be provided at a position such as an arm, abdomen or thigh.

In some examples, the blood of the object 2 may also be collected through a blood collection needle (for example, the blood is collected from a fingertip, palm, or arm), and a target physiological parameter in the finger blood may be measured using the blood glucose meter 10b. Optionally, the blood (finger blood) of the fingertip of the object 2 may be collected. In this case, the fingertip has a highly dense capillary network, the change of blood glucose concentration in the body may be quickly reflected, thus facilitating control of the blood glucose concentration.

In some examples, referring to FIG. 5, the target physiological parameter of the object 2 at a plurality of time nodes and the residual activity parameter of the chemical substance may be acquired. The plurality of time nodes may be continuous time nodes. In some examples, the plurality of time nodes include a target node and other nodes, and residual activity parameter of the target node are obtained based on residual activity parameters of the other nodes. In this case, parameters such as the residual activity parameter, the increase rate coefficient, or the sensitivity coefficient will not change too much in a short time. When parameters such as a residual activity parameter, an increase rate coefficient, or a sensitivity coefficient of one time node are calculated, the accuracy of calculation may be improved using parameters of multiple time nodes.

In some examples, referring to FIG. 5, other nodes may be nodes before the target node. For example, the target node may be a k+1^{th} time node, and the other nodes may be a k^{th} time node and a k−1^{th} time node. It should be noted that an identical time node may be a target node or one of other nodes. Specifically, one time node is allowed to correspond to one control step. In one control step, a fifth time node may be the target node, and a fourth time node and a third time node may be the other nodes. In a next control step, a sixth time node may be the target node, and a fifth time node and a fourth time node may be the other nodes.

In some examples, in one control step, the time nodes may include not less than two other nodes and one target node. Optionally, in one control step, the time nodes may include two other nodes and one target node. In this case, a large number of other nodes may reflect a complex algorithm model. A large number of other nodes may reduce the complexity of the algorithm and reduce the calculation cost.

In some examples, the residual activity parameter may satisfy the metabolic model described above:

$${I}_{eff} ( k + 1 ) = {K}_{0} {I}_{s c} ( k ) + {K}_{1} {I}_{eff} ( k ) - {K}_{2} {I}_{eff} ( k - 1 ) ,$$

In this case, the active insulin of the target node may be obtained based on the insulin infusion dose of the insulin, and the active insulin of the other nodes.

### (Step S105)

In some examples, step S105 may be calculating the sensitivity coefficient of the chemical substance based on the target physiological parameter and the residual activity parameter.

In some examples, the sensitivity coefficient may be used for characterizing the influence level of the chemical substance on the target physiological parameter. In a case where the chemical substance is the insulin, the sensitivity coefficient may also be called the insulin sensitivity coefficient. In some examples, the sensitivity coefficient is a negative. The smaller the sensitivity coefficient is, the greater the influence level of the chemical substance on the target physiological parameter is. The greater the sensitivity coefficient (closer to 0) is, the smaller the influence level of the chemical substance on the target physiological parameter is. In other words, the greater the absolute value of the sensitivity coefficient is, the greater the influence level of the chemical substance on the target physiological parameter is. The smaller the absolute value of the sensitivity coefficient is, the smaller the influence level of the chemical substance on the target physiological parameter is.

In some examples, the sensitivity coefficient may be obtained through formula iteration. In this case, the sensitivity coefficient at each time node may be obtained using formula iteration so as to obtain an adaptively adjusted sensitivity coefficient, resulting in improving the control accuracy.

In some examples, the initial value of the sensitivity coefficient may be actively input or calculated by the processing apparatus 30 based on the preset parameters. In some examples, the initial value of the sensitivity coefficient may be obtained through the ways such as rule 500, rule 1500 or rule 1800. In this case, the initial value of the sensitivity coefficient may be conveniently obtained, and then the sensitivity coefficient at each time node may be obtained using the formula iteration.

In some examples, a calculation formula of the sensitivity coefficient may be obtained through loss functions. Specifically, the sensitivity coefficient located at the target node may be obtained through the way of minimizing loss function. The loss function may include an error term and a regularization term. The error term is used for characterizing a difference between the target physiological parameter acquired by measurement and the target physiological parameter acquired by calculation. The regularization term is used for characterizing a difference degree of the sensitivity coefficients of adjacent time nodes. In this case, the accuracy of the sensitivity coefficient of each time node may be improved using an error term. Meanwhile, since the sensitivity coefficient will not change too much in a short time under a normal circumstance, the stability and accuracy of the sensitivity coefficient may also be improved using the regularization term.

In some examples, the sensitivity coefficient of the target node may be acquired based on the sensitivity coefficients of the other nodes, the target physiological parameters of the other nodes, the residual activity parameters of the other nodes, and the target physiological parameter of the target node. In this case, the sensitivity coefficient of the target node may be calculated using some of parameters acquired in a previous time node, thereby realize adaptive adjustment of the sensitivity coefficient. Meanwhile, since the sensitivity coefficient will not change too much in the adjacent time nodes, the stability and accuracy of the sensitivity coefficient may be improved using the adjacent time nodes.

Hereinafter the acquisition way of the sensitivity coefficient is further explained. In some examples, the loss function may satisfy the formula:

$$L ( φ ) = {\vert G ( k ) - G ( k - 1 ) - α ( k - 1 ) - φ ( k ) {I}_{eff} ( k - 1 ) \vert}^{2} + {μ ( φ ( k ) - \hat{φ} ( k - 1 ) )}^{2}$$

Wherein L(φ) may represent the loss function, G(k) may represent the target physiological parameter (i.e., the blood glucose concentration) at the k^{th} time node, α(k−1) may represent an increase rate coefficient at the k−1^{th} time node, φ(k) may represent the sensitivity coefficient of the chemical substance at the k^{th} time node (i.e., the insulin sensitivity coefficient), I_{eff} (k−1) may represent the residual activity parameter (i.e., the active insulin) of the chemical substance at the k−1^{th} time node, μ may represent a regularization term coefficient, which is used for adjusting an action ratio of the error term and the regularization term, \hat{φ}(k−1) may represent a predicted value of the sensitivity coefficient of the chemical substance at the k−1^{th} time node (i.e., the predicted value of the insulin sensitivity coefficient), |G(k)−G(k−1)−α(k−1)−φ(k)I_{eff}(k−1)|² may represent the error term and μ(φ(k)−\hat{φ}(k−1))² may represent the regularization term.

It should be noted that the error term and the form of the error term are not limited to this, an expression that may be used for characterizing a difference between the target physiological parameter acquired by measurement and the target physiological parameter acquired by calculation may be used as the error term, for example, expressions for a ratio, a difference value or an absolute value (or a plurality of powers such as square and cube) of the difference value between the target physiological parameter acquired by measurement and the target physiological parameter acquired by calculation may be used as the error term, while expressions that may be used for characterizing a degree of difference of the sensitivity coefficients of adjacent time nodes may also be used as the regularization term, for example, expressions for a ratio, a difference value or an absolute value (or a plurality of powers such as square and cube) of the difference value between the sensitivity coefficients of the adjacent time nodes may be taken as the regularization term.

In some examples, the regularization term coefficients may be set empirically or may be default data set by the processing apparatus 30.

In some examples, the predicted value of the sensitivity coefficient of chemical substance may satisfy the formula:

$$\hat{φ} ( k ) = arg min ( L ( φ ) ) ,$$

Wherein arg min(L(φ)) represents that L(φ) is allowed to be minimized.

In some examples, a first iteration formula of the sensitivity coefficient may be obtained based on the formula satisfied by the predicted value of the sensitivity coefficient of the chemical substance. In some examples, the first iteration formula of the sensitivity coefficient may be obtained by derivation of L(φ). In some examples, an iteration step size in the first iteration formula may be set empirically.

In some examples, the sensitivity coefficient acquired by calculation (i.e., the predicted value of the sensitivity coefficient) may be taken as sensitivity coefficients of other steps for calculation.

### (Step S107)

In some examples, step S107 may be calculating the increase rate coefficient of the target physiological parameter based on the target physiological parameter and the residual activity parameter. In some examples, the increase rate coefficient may be used for characterizing the influence level of factors other than the chemical substance on the target physiological parameter. When the chemical substance is the insulin, the increase rate coefficient may also be called a blood glucose increase rate. The increase rate coefficient may be used for characterizing an influence level of factors such as carbohydrate intake, endogenous glucose secretion, or an unexplained change in insulin sensitivity on the blood glucose concentration. In this case, since the increase rate coefficient may be used to reflect effects of various factors, and the calculation of the increase rate coefficient does not require the input of carbohydrate (described later), the input of the parameters may be reduced, and meanwhile, the accuracy of the model may be improved.

In some examples, the increase rate coefficient of the target node may be obtained based on the difference between the target physiological parameter acquired by measurement and the target physiological parameter acquired by calculation located at the target node, and the increase rate coefficients of the other nodes. In this case, the increase rate coefficient of the target node may be calculated using some of parameters acquired in a previous time nodes, thereby adaptively adjusting the increase rate coefficient, so that the accuracy of the sensitivity coefficient may be improved.

Hereinafter the acquisition way of the increase rate coefficient is further explained. In some examples, the increase rate coefficient may satisfy the formula:

$$\hat{α} ( k ) = G ( k ) - \hat{G} ( k ) + \hat{α} ( k - 1 ) = e ( k ) + \hat{α} ( k - 1 ) ,$$

Wherein \hat{α}(k) may represent a predicted value of the increase rate coefficient at the k^{th} time node, Ĝ(k) may represent a calculated value of the blood glucose concentration at the k^{th} time node (i.e., a predicted value of the blood glucose concentration), and ê(k) may represent a difference between the predicted value of the blood glucose concentration at the k^{th} time node and a measured value of the blood glucose concentration.

It should be noted that the predicted value of the blood glucose concentration and the measured value of the blood glucose concentration may be understood in the following way: the predicted value Ĝ(k) of the blood glucose concentration is an intermediate parameter appearing in step S109, which may be obtained based on the blood glucose concentration at the k−1^{th} time node, the predicted value of the insulin sensitivity coefficient, the active insulin, the increase rate coefficient and the blood glucose dynamics model, and the measured value of the blood glucose concentration is the blood glucose concentration obtained by the continuous blood glucose monitoring apparatus 10a or blood glucose meter 10b described above. Unless otherwise specified, the blood glucose concentration in the body of the object 2 and the blood glucose concentration involved in a derivation process in other steps may refer to the measured value of the blood glucose concentration.

In some examples, referring to simplified results of the formula that the increase rate coefficient satisfies. The increase rate coefficient may satisfy a second iteration formula:

$$\hat{α} ( k ) = e ( k ) + \hat{α} ( k - 1 ) ,$$

In other words, the increase rate coefficient may represent the sum of the differences between the predicted value of the blood glucose concentration and the measured value of the blood glucose concentration in different control steps (i.e., in different time nodes).

In some examples, the initial value of the increase rate coefficient may be 0, but the present disclosure is not limited to this. The increase rate coefficient may also be any value.

### (Step S109)

In some examples, in step S109, the safe dose and the basal dose of the chemical substance may be calculated based on the preset parameters, the target physiological parameter, the residual activity parameter, the sensitivity coefficient and increase rate coefficient. The safe dose may refer to a dose that the target physiological parameter will not be lower than the safety threshold value in a long time after the chemical substance is infused into the object 2. The basal dose may refer to a dose that the target physiological parameter may reach a target value in a short time (such as one control step) after the chemical substance is infused into the object 2. In this case, after the infusion of the chemical substance, there will be the chemical substance remaining active at each time node in a metabolic process of the chemical substance. Under the action of the chemical substance remaining active at a plurality of time nodes, the adjustment of the target physiological parameter will overlap, which may cause the target physiological parameter to exceed the safety range (for example, below the safety threshold value), while the safe dose and the basal dose are calculated at the same time, so that a difference between two kinds of doses may be determined, which may then guarantee the safety and adjustment effect of the infusion dose.

In some examples, in a process of calculating the basal dose, according to the blood glucose dynamics model, the residual activity parameter of the chemical substance may satisfy the formula:

$${I}_{eff} ( k ) = \frac{( {G}_{d} ( k + 1 ) - G ( k ) - α ( k ) )}{φ ( k )}$$

Wherein G_{d} (k+1) may represent the target value of the target physiological parameter at the k+1^{th} time node. In some examples, the target value may be obtained via calculation. For example, the target value may be obtained through calculation of the target physiological parameter at the k^{th} time node and the control step size. In some examples, the target value may also be obtained through manual input.

In some examples, in a process of calculating the basal dose, according to the blood glucose dynamics model, the residual activity parameter of the chemical substance may also satisfy the formula:

$${I}_{eff} ( k ) = \frac{ρ ( {G}_{d} ( k + 1 ) - G ( k ) - α ( k ) )}{λ + φ ( k )}$$

Wherein λ, ρ may represent adjustment parameters. In this case, since there may be a possibility that φ approaches to 0, resulting in a large degree of change of I_{eff}(k). I_{eff}(k) can be stabilized by addition of the adjustment parameters, so that the subsequent calculated basal dose may be controlled within an appropriate range. In some examples, the adjustment of parameters may be selected or adjusted empirically.

In some examples, the basal dose may be obtained according to the metabolic model of the insulin and the calculation formula of the residual activity parameter of the chemical substance.

In some examples, in some examples, the adjustment parameters ρ may be used to adjust the basal dose. For example, in a case where the adaptive closed-loop control method is used to adjust the blood glucose concentration, identical infusion dose may have different influence levels on adults and children. A safe and appropriate dose for the adults may have significant impact on the children (for example, causing hypoglycemia). Therefore, the dose for the children may be reduced in a certain proportion, so that a value range of ρ may be set to be from 0 to 1, and different ρ is set as to different populations. In this case, adaptability of the adaptive closed-loop control method to different populations may be improved.

In some examples, in a process of calculating the safe dose, the continuous effect of the chemical substance remaining active on the target physiological parameter may be calculated first. Taking the insulin as an example, the change of future active insulin volume over time may be predicted based on the metabolic model of the insulin and historical insulin infusion information.

In some examples, the future active insulin and the insulin sensitivity coefficient may be used to obtain the total impact of the insulin infused into the object 2 on the blood glucose concentration. Specifically, after the insulin is infused into the object 2 at the k−1^{th} time node, the impact of active insulin on the blood glucose concentration may be integrated to obtain the total impact on the blood glucose concentration.

In some examples, when the total impact of insulin infusion at the k−1^{th} time node on the blood glucose concentration, the sensitivity coefficient may be a constant. In this case, since one control step corresponds to one time node, each control step may calculate the total impact of the active insulin on the blood glucose concentration, and therefore, the blood glucose dynamics model established in a single control step may be appropriately simplified, while the total impact of the active insulin on the blood glucose concentration is calculated in one control step, the sensitivity coefficient is a constant, which may reduce the calculation cost and increase the calculation speed.

In some examples, in a process of calculating the safe dose, the safe dose may be obtained according to the total effect of the active insulin on the blood glucose concentration and the blood glucose dynamics model, without considering the increase rate coefficient.

In some examples, in a process of calculating the safe dose, PID (proportional integral derivative) control principle may also be added in a case where the increase rate coefficient is considered. Specifically, the increase rate coefficient may be used to construct a PID term. In this case, the accuracy and stability of the adaptive closed-loop control method may be improved.

### (Step S111)

In some examples, in step S111, the target dose of the chemical substance may be calculated based on the safe dose and the basal dose. In this case, calculation results of the safe dose and basal dose may be considered at the same time and the appropriate target dose may be obtained.

In some examples, the smaller one in the safe dose and the basal dose may be taken as the target dose. In this case, the safety of the adaptive closed-loop control method may be improved.

In some examples, as described above, the chemical substance of the infusion dose may be allowed to enter the object 2 by infusion. The infusion dose is related to the target dose and the infusion accuracy. In some examples, rounding down may be used to calculated the infusion dose. For example, in the infusing device 20, the infusion accuracy is 0.1 U/time, and the calculated target dose is 6.47 U, the infusion dose may be 6.4 U. In this case, the calculated target dose may cooperate with actual infusion accuracy of an actual device to obtain the infusion dose that may be infused to the object 2 within an accuracy range.

In some examples, the infusion dose may be obtained through metering with the infusing device 20. In this case, due to uncertainty of actual infusion, metering of the infusing device 20 is used to obtain the infusion dose, capable of obtaining the actual dose of the chemical substance infused into the object 2 more accurately, facilitating to improve the accuracy of subsequent regulation.

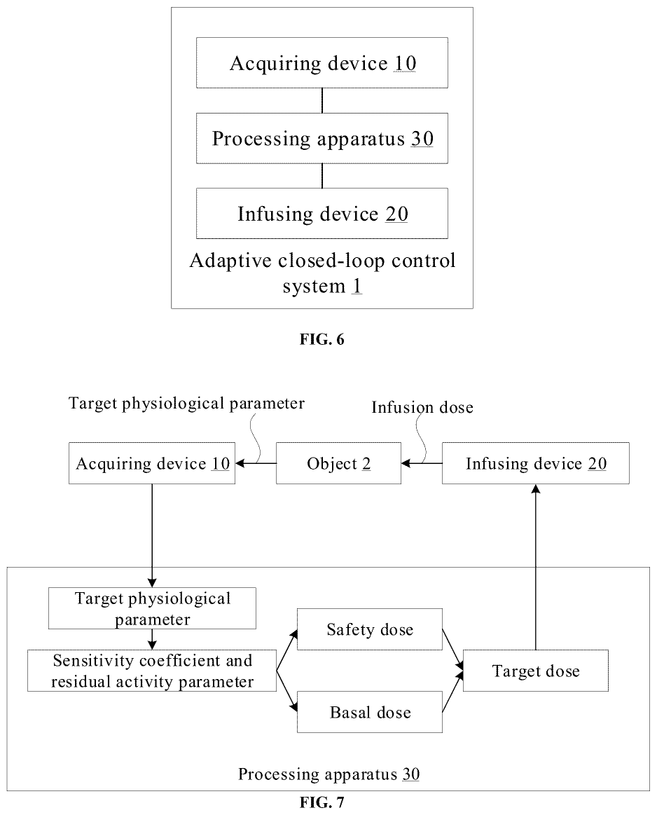

FIG. 6 is a structural block diagram illustrating a linear model-based adaptive closed-loop control system 1 according to an embodiment of the disclosure. FIG. 7 is a flow chart illustrating a linear model-based adaptive closed-loop control system 1 according to an embodiment of the disclosure.

As described above, the present disclosure further relates to a linear model-based adaptive closed-loop control system 1, using the adaptive closed-loop control method according to an embodiment of the disclosure to control a target physiological parameter of an object 2. In this case, a physiological parameter related to the target physiological parameter may be adaptively adjusted, the stability of the target physiological parameter of the object 2 may be improved, and input parameters may be reduced.

In some examples, referring to FIG. 6, the linear model-based adaptive closed-loop control system 1 may include an infusing device 20, a processing apparatus 30, and an acquiring device 10.

In some examples, referring to FIG. 7, the acquiring device 10 may be used to acquire the target physiological parameter of the object 2.

In some examples, referring to FIG. 7, the infusing device 20 may infuse the chemical substance into the object 2 to adjust the target physiological parameter of the object 2.

In some examples, referring to FIG. 7, the processing apparatus 30 may calculate the dose of the chemical substance required to be infused by the infusing device 20, based on preset coefficients input earlier and the target physiological parameter acquired by the acquiring device 10.

In some examples, referring to FIG. 7, after acquiring the target physiological parameter, the processing apparatus 30 may calculate the sensitivity coefficient and the residual activity parameter, calculate the safe dose and basal dose based on the sensitivity coefficient and the residual activity parameter, calculate the target dose based on the safe dose and the basal dose, and send the target dose to the infusing device 20.

In some examples, referring to FIG. 7, the infusing device 20 may infuse the chemical substance of infusion dose to the object 2 based on the target dose.

In some examples, the processing apparatus 30 may use the adaptive closed-loop control method according to an embodiment of the disclosure to obtain the target dose, and control the infusing device 20 to infuse the chemical substance.

### (Simulation Results)

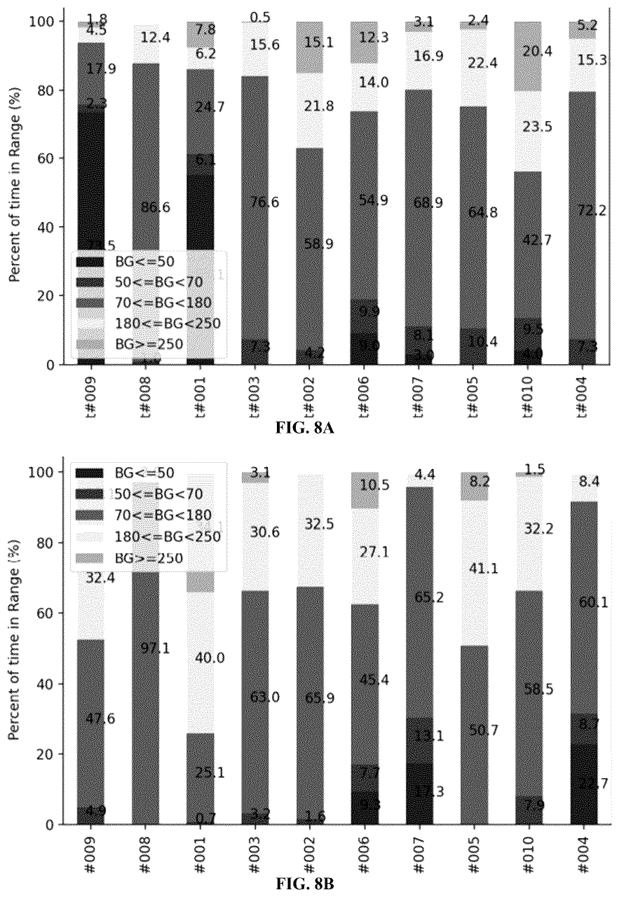

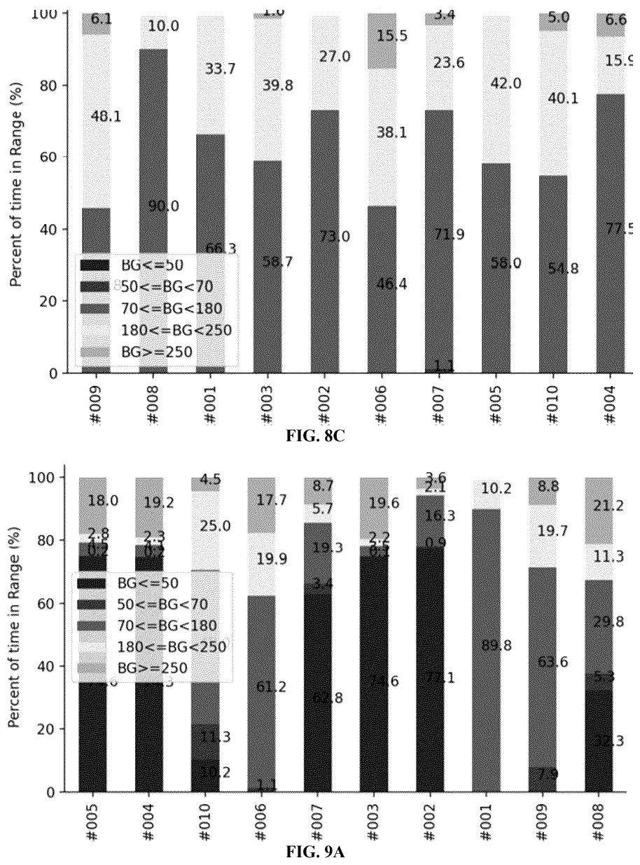

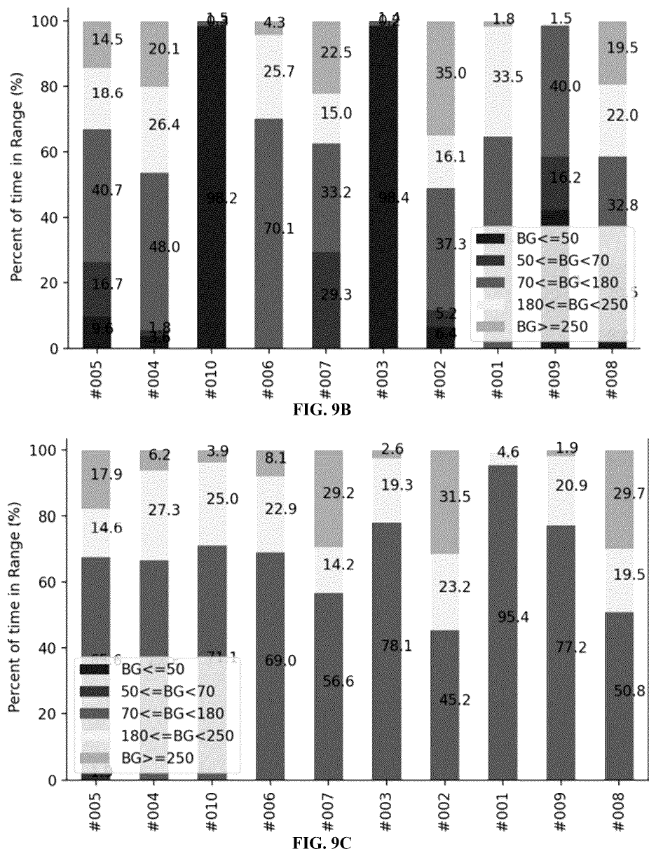

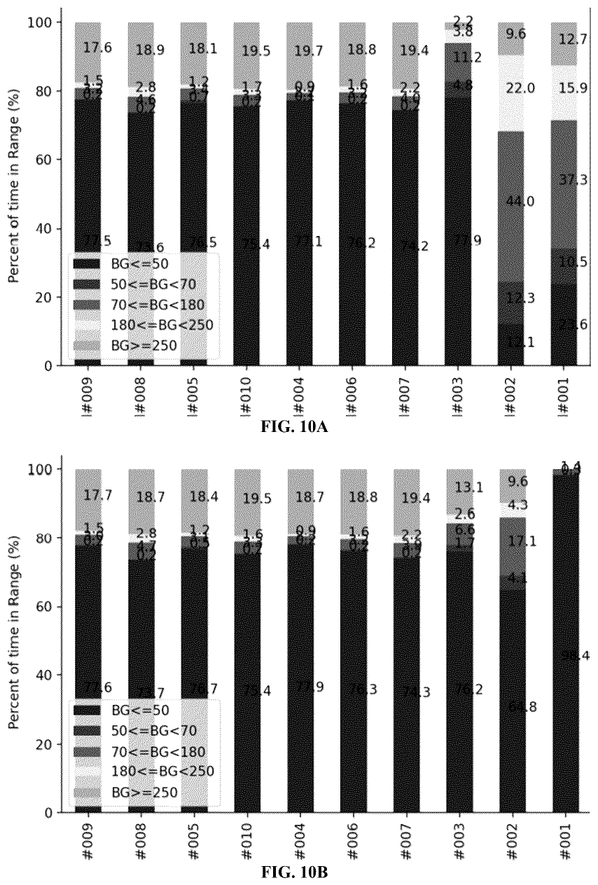

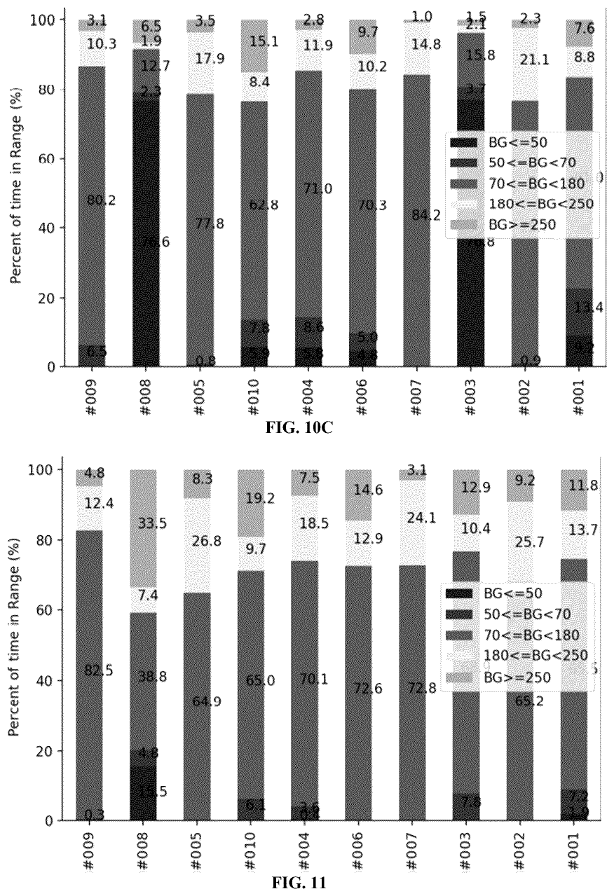

FIG. 8a is a simulation schematic diagram illustrating that a plain PID method is applied to an adult according to an embodiment of the disclosure. FIG. 8b is a simulation schematic diagram illustrating that a PIDIFB method is applied to an adult according to an embodiment of the disclosure. FIG. 8c is a simulation schematic diagram illustrating that an adaptive closed-loop control method is applied to an adult according to an embodiment of the disclosure. FIG. 9a is a simulation schematic diagram illustrating that a plain PID method is applied to an adolescent according to an embodiment of the disclosure. FIG. 9b is a simulation schematic diagram illustrating that a PIDIFB method is applied to an adolescent according to an embodiment of the disclosure. FIG. 9c is a simulation schematic diagram illustrating that an adaptive closed-loop control method is applied to an adolescent according to an embodiment of the disclosure. FIG. 10a is a simulation schematic diagram illustrating that a plain PID method is applied to a child according to an embodiment of the disclosure. FIG. 10b is a simulation schematic diagram illustrating that a PIDIFB method is applied to a child according to an embodiment of the disclosure. FIG. 10c is a simulation schematic diagram illustrating that an adaptive closed-loop control method is applied to a child according to an embodiment of the disclosure.

The simulation schematic diagrams are diagrams obtained through a Simglucose model. Simulation objects include adults, adolescents and children. Dexcom's and Insulet's continuous glucose monitoring apparatuses 10a (CGM) are used in the simulation. The initial value of the insulin sensitivity coefficient may be set by rule 1800.

In some examples, the plain PID method may refer to a control method that takes historical blood glucose data and insulin injection data as input, without stating the carbohydrate, and simply uses PID. The PIDIFB method may be a control method combining the PID method with insulin feedback. LMAPID may refer to the adaptive closed-loop control method according to embodiments of the disclosure.

Referring to FIG. 8a to FIG. 10c, whether adults, adolescents or children, compared with the plain PID method and the PIDIFB method, the adaptive closed-loop control method according to an embodiment of the disclosure has better simulation results.

Referring to FIG. 10c, when the adaptive closed-loop control method is applied to the children, there is a risk of hypoglycemia in the simulation results of the objects 2 numbered #008 and #003. In this regard, the adaptive closed-loop control method may be optimized. For example, the target dose of the insulin may be reduced in an equal proportion for the children. Specifically, the target dose may be reduced by 50% to 90% in an equal proportion. In this case, the risk of hypoglycemia may be lowered.

FIG. 11 is a simulation schematic diagram illustrating that an optimized adaptive closed-loop control method is applied to a child according to an embodiment of the disclosure.

Referring to FIG. 11, the optimized adaptive closed-loop control method (the target dose is reduced by 70% in an equal proportion) effectively lowers the risk of hypoglycemia.

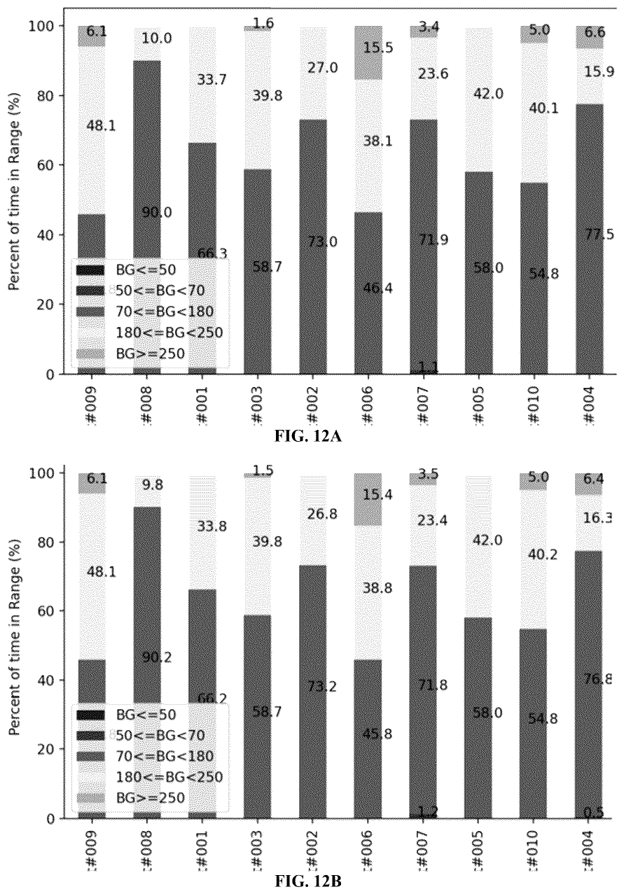

FIG. 12a is a simulation schematic diagram illustrating that an adaptive closed-loop control method with infinite infusion accuracy is applied to an adult according to an embodiment of the disclosure. FIG. 12b is a simulation schematic diagram illustrating that an adaptive closed-loop control method with finite infusion accuracy is applied to an adult according to an embodiment of the disclosure.

It should be noted that in the adaptive closed-loop control method with the infinite infusion accuracy, the target dose may be the same as the infusion dose. In the adaptive closed-loop control method with the finite infusion accuracy, the method described above based on the infusion accuracy and the target dose may be used to calculate the infusion dose. FIG. 12b represents that the infusion accuracy is 0.1 U/time. Referring to FIG. 12a and FIG. 12b, the simulation results of the adaptive closed-loop control method with the infinite infusion accuracy are roughly the same as those of the adaptive closed-loop control method with infusion accuracy of 0.1 U/time, which indicates that the adaptive closed-loop control method according to embodiments of the disclosure is less sensitive to the infusion accuracy within a certain accuracy range. In other words, within the certain accuracy range, even if the adaptive closed-loop control method according to embodiments of the disclosure is used in the infusing device 20 with relatively low accuracy, a relatively good control effect still may be kept.

While the present disclosure has been specifically described above in combination with the drawings and embodiments, it is understood that the above description does not limit the present disclosure in any form. Those skilled in the art may, as required, make changes to the present disclosure without departing from the essential spirit and scope of the present disclosure, and these changes fall within the scope of the present disclosure.

Various embodiments of the disclosure may have one or more of the following effects. In some embodiments, a physiological parameter related to the target physiological parameter may be adaptively adjusted, and input parameters are reduced. In other embodiments, the disclosure may provide a closed-loop control method that controls a target physiological parameter of an object using a chemical substance. In further embodiments, the disclosure may provide a linear model-based adaptive closed-loop control method that may reduce input parameters and meanwhile may adaptively adjust a physiological parameter related to a target physiological parameter may be provided. The present disclosure may be completed in view of a state of the prior art, and one of its purposes may be to provide a linear model-based adaptive closed-loop control method that may reduce input parameters and meanwhile may adaptively adjust a physiological parameter related to a target physiological parameter.

Many different arrangements of the various components depicted, as well as components not shown, are possible without departing from the spirit and scope of the disclosure. Embodiments of the disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

1.-10. (canceled)

11. A linear model-based adaptive closed-loop control method, which is a control method to control a target physiological parameter of an object using a chemical substance, characterized by comprising:
acquiring preset parameters;
acquiring a target physiological parameter of the object and a residual activity parameter of the chemical substance;
calculating a sensitivity coefficient of the chemical substance based on the target physiological parameter and the residual activity parameter, the sensitivity coefficient being used for characterizing an influence level of the chemical substance on the target physiological parameter;
calculating increase a rate coefficient of the target physiological parameter based on the target physiological parameter and the residual activity parameter, the increase rate coefficient being used for characterizing an influence level of factors other than the chemical substance on the target physiological parameter;
calculating a safe dose and a basal dose of the chemical substance based on the preset parameters, the target physiological parameter, the residual activity parameter, the sensitivity coefficient and the increase rate coefficient; and
calculating a target dose of the chemical substance based on the safe dose and the basal dose.

12. The adaptive closed-loop control method according to claim 11, wherein the preset parameters comprise a physiological information of the object, a type of the chemical substance, a control step size, a unit of the target physiological parameter, a safety threshold value of the target physiological parameter, and an infusion accuracy of the chemical substance.

13. The adaptive closed-loop control method according to claim 11, wherein the preset parameters are obtained data import or manual input.

14. The adaptive closed-loop control method according to claim 11, wherein the chemical substance of an infusion dose is enabled to enter an object infusion, the infusion dose being related to the target dose and an infusion accuracy of the chemical substance.

15. The adaptive closed-loop control method according to claim 14, wherein the infusion dose is obtained by metering of an infusing device.

16. The adaptive closed-loop control method according to claim 11, wherein the target physiological parameter is collected by a continuous blood glucose monitoring apparatus or a blood glucose meter.

17. The adaptive closed-loop control method according to claim 11, wherein:
the target physiological parameters of the object at a plurality of time nodes and the residual activity parameter of the chemical substance are acquired;
the plurality of time nodes include a target node and other nodes; and
the residual activity parameter of the target node is obtained based on the residual activity parameters of the other nodes.

18. The adaptive closed-loop control method according to claim 17, wherein the other nodes are nodes before the target node.

19. The adaptive closed-loop control method according to claim 17, wherein the plurality of time nodes include two other nodes and one target node.

20. The adaptive closed-loop control method according to claim 17, wherein:
the sensitivity coefficient located at the target node is obtained by minimizing a loss function;
the loss function comprises an error term and a regularization term;
the error term is used for characterizing a difference between the target physiological parameter acquired by measurement and the target physiological parameter acquired by calculation; and
the regularization term is used for characterizing a difference degree of the sensitivity coefficients of adjacent time nodes.

21. The adaptive closed-loop control method according to claim 20, wherein the sensitivity coefficient of the target node is acquired based on the sensitivity coefficients of the other nodes, the target physiological parameters of the other nodes, the residual activity parameters of the other nodes, and the target physiological parameter of the target node.

22. The adaptive closed-loop control method according to claim 20, wherein an initial value of the sensitivity coefficient is actively input or calculated by a processing apparatus based on the preset parameters.

23. The adaptive closed-loop control method according to claim 17, wherein the increase rate coefficient of the target node is obtained based on a difference between the target physiological parameter acquired by measurement and the target physiological parameter acquired by calculation located at the target node, and the increase rate coefficients of the other nodes.

24. The adaptive closed-loop control method according to claim 23, wherein the increase rate coefficient at a k^{th} time node satisfies a second iteration formula:
$$\hat{α} ( k ) = e ( k ) + \hat{α} ( k - 1 ) ,$$
wherein \hat{α}(k) represents a predicted value of the increase rate coefficient at the k^{th} time node, e(k) represents a difference between the target physiological parameter acquired by measurement, and the target physiological parameter acquired by calculation at the k^{th} time node.

25. The adaptive closed-loop control method according to claim 23, wherein an initial value of the increase rate coefficient is 0.

26. The adaptive closed-loop control method according to claim 11 wherein a smaller one in the safe dose and the basal dose is taken as the target dose.

27. The adaptive closed-loop control method according to claim 11 wherein:
the basal dose is obtained according to a metabolic model of the chemical substance and a calculation formula of the residual activity parameter of the chemical substance; and
the calculation formula of the residual activity parameter of the chemical substance is obtained according to a dynamics model of the target physiological parameter.

28. The adaptive closed-loop control method according to claim 27 wherein the dynamics model is expressed as:
$$Δ G ( k + 1 ) = G ( k + 1 ) - G ( k ) = φ {I}_{eff} + α ,$$
wherein ΔG(k+1) represents a change of the target physiological parameter at a k+1th time node, G(k+1) represents the target physiological parameter at the k+1th time node, φ represents the sensitivity coefficient of the chemical substance, I_{eff} represents the residual activity parameter of the chemical substance, and α represents the increase rate coefficient of the target physiological parameter.

29. The adaptive closed-loop control method according to claim 11 wherein:
the target physiological parameter is a blood glucose concentration;
the chemical substance is insulin; and
the sensitivity coefficient is an insulin sensitivity coefficient.

30. A linear model-based adaptive closed-loop control system, wherein a target physiological parameter of an object is controlled using the adaptive closed-loop control method in claim 11.
